(19) United States
(12) Patent Application Publication
(10) Pub. No.: US 2002/0156303 A1
(43) Pub. Date: Oct. 24, 2002

(54) Process for the preparation of conjugated estrogens from pregnant mare urine

(75) Inventors: Vallapa Soong, Hsien (TW); Jing-Yi Wang, Tainan (TW)

Correspondence Address: FINNEGAN, HENDERSON, FARABOW, GARRETT &, DUNNER LLP, 1300 I STREET, NW, WASHINGTON, DC, 20005, US

(73) Assignee: SCINOPHARM TAIWAN, LTD.

(21) Appl. No.: 09/837,227

(22) Filed: Apr. 19, 2001

(51) Int. Cl. C07J 1/00

(52) U.S. Cl. 552/625

(57) ABSTRACT

This invention is directed to a process for the preparation of conjugated estrogens from pregnant mare urine (PMU), comprising the steps of

(a) treating PMU material with an alkaline solvent;

(b) filtrating the treated PMU material to remove mucilaginous substances and solids;

(c) contracting the filtered PMU resultant with a sufficient quantity of polystyrene absorber resins; and

(d) eluting the resins with a water-miscible solvent to separate conjugate estrogens and resins and then obtaining crude extract conjugated estrogens.

### FIELD OF THE INVENTION

This invention is directed to a process for the preparation of conjugated estrogens from pregnant mare urine (PMU).

### BACKGROUND OF THE INVENTION

Conjugated estrogens are effective drugs for treating the symptoms of menopause. Conjugated estrogens coming from a natural source such as pregnant mare urine (PMU) are found to be particularly effective.

The extracts containing conjugated estrogens can be obtained from the PMU by extraction with a polar organic solvent. However, such liquid-liquid extractions cause a number of problems such as foaming, sedimentation, emulsification and poor phase separation. Therefore, a solid-liquid extraction method has been developed for the extraction of conjugated estrogens from PMU.

H. L. Bradlow etc., Steroids, 11(1968), pp. 265-272, discloses that Amberlite® XAD-2 (a styrene-divinylbenzene copolymer, supplied by Rohm & Haas) was proposed to be useful in the extraction of conjugated estrogens from urine. However, the adsorption capacity obtained is low. By using Bradlow's method, the combined residue from extraction usually contains 10% or less of the urine solids.

U.S. Pat. No. 3,769,401, issued to Thompson L. on Oct. 30, 1973, suggests using Dowex 1-X2-C1 for the extraction of conjugated estrogens from urine, which is a strong basic anion exchange resin containing quaternary ammonium functional groups which are attached to a styrenedivinylbenzene copolymer.

DEAE-Sephadex® (diethylaminoethyl, DEAE Sephadex®) was found to be useful in the separation of conjugated estrogens in 1965 by Hahnel, R., Anal Biochem and 1969 by Hobkirk, R., Musey, P. I. and Nilsen, M., Steroids. However, the results showed that a salt gradient is necessary for the elution of estrogen conjugates. P. I. Musey etc., May 1997, Steroids, Vol.29, No.5, pp.657-668 created an isocratic method for the separation of estrogen conjugates, instead of the previous chromatographic separation, to obtain a higher degree of resolution without using a gradient system. DEAE-Sephadex can be purchased from Pharmacial Piscataway.

U.S. Pat. No. 5,723,454, issued to Ivan Ban et al. on Mar. 3, 1998, discloses a method for obtaining an extract containing the natural mixture of conjugated estrogens from pregnant mare urine by solid-phase extraction of the mixture on non-ionic semi-polar polymeric adsorber resins. The method comprises steps of filtering PMU by microfiltration, contacting Amberlite XAD-7™, washing with NaOH to pH in the range from 12.5 to 13.5, and eluting with EtOH:water (30:70) to pH about 12. U.S. Pat. No. 5,723,454 uses Amberlite XAD-7™ as a semi-polar polymeric adsorber resin, which is a macroporous cross-linked aliphatic polycarboxylic ester manufactured by Rohm and Haas.

U.S. Pat. No. 5,814,624, issued to Ivan Ban et al. on Sep. 29, 1998, discloses a method for obtaining an extract from urine of pregnant mares containing a natural mixture of conjugated estrogens by solid-phase extraction of the mixture on reverse-phase (RP) silica gel. The method comprises complex steps of filtering PMU with a sand bed or microfiltration apparatus, adjust pH value to about 13 with NaOH and then about 8 to 8.5 with HCl, contacting RP-silica gel, washing with acetic/sodium acetate buffer, and washing with EtOH-water mixture. U.S. Pat. No. 5,814,624 illustrates Kieselgel 60/dimethylsilane derivative manufactured by Merck as a RP silica gel.

Although the developed solid-phase extractions overcome most of the disadvantages caused by liquid-liquid extraction, some problems still remain. For example, the process is time-consuming; the resin used has a limited shelf life and high cost; the viscosity of the starting materials treated with the adsorber resin is too high for a large scale process; and the total recovery rate of the conjugated estrogen is low, generally, less than 80%.

Therefore, there is a need in the art for a more effective high yield way to recover estrogens from pregnant mare urine.

### SUMMARY OF THE INVENTION

The present invention provides a process for the preparation of conjugated estrogens from pregnant mare urine (PMU) resulting in a very high recovery rate of conjugated estiogens, while avoiding the disadvantages known from the conventionally used liquid-liquid and solid-liquid extractions.

The object of the present invention is achieved by a process for the preparation of conjugated estrogens from pregnant mare urine (PMU), comprising the steps of (a) treating PMU material with an alkaline solvent; (b) filtrating the treated PMU material to remove mucilaginous substances and solids; (c) contacting the filtered PMU resultant with a sufficient quantity of polystyrene absorber resins; and (d) eluting the resins with a water-miscible solvent to separate conjugated estrogens and resins and then obtaining crude extract conjugated estrogens.

### DETAILED DESCRIPTION OF THE INVENTION

The present invention provides a process for the preparation of conjugated estrogens from pregnant mare urine (PMU), comprising the steps of (a) treating PMU material with an alkaline solvent; (b) filtrating the treated PMU material to remove mucilaginous substances and solids; (c) contacting the filtered PMU resultant with a sufficient quantity of polystyrene absorber resins; and (d) eluting the resins with a—water-miscible solvent to separate conjugate estrogens and resins and then obtaining crude extract conjugated estrogens.

The pH value of originial PMU is generally in the range from 7 to 8. In step (a) of the process, a quantity of an alkaline solvent sufficient to provide a pH value in the range of about 9 to 12, preferably 9 to 11, is added to PMU to reduce the viscosity of PMU, removing some lactone and phenolic material such as phenolic acid, and eliminating urine odor. The amount of alkaline solvent can be varied depending on its concentration and the mount of PMU. Suitable alkaline solvents include alkali metal or alkaline earth metal hydroxides and carbonates. Preferred alkaline solvents include sodium hydroxide, potassium hydroxide, sodium carbonate and potassium carbonate. Most preferred alkaline solvent is sodium hydroxide, which results in the recovery rate of conjugated estrogens to about 100%.

In step (b) of the process, the PMU material treated with the alkaline solvents from step (a) is filtered to remove the mucilaginous substances and solids. According to the invention, the filtration is preferably carried out by centrifugation.

In step (c) of the process, the filtered PMU resultant from step (b) is subjected to contact with a sufficient amount of polystyrene absorber resins to absorb the conjugated estrogens. The polystyrene absorber resins preferably have a surface area in the range of 500-1500 m²/g, a pore volume in the range of 1.2-2.5 ml/g, and a pore area in the range of 80-350 Å. The term “polystyrene” means a polymer containing the monomer of styrene (phenylethylene). Typically, polystyrene absorber resins contain the moiety of following structural formula:

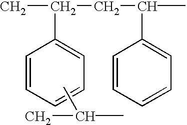

Suitable polystyrene absorber resins for the present invention are such as Diaion HP-20 and Sepabeads SP-700, manufactured by Mitsubishi. Diaion HP-20 has a surface area of about 600 m²/g, a pore volume of about 1.5 ml/g, and a pore area in the range from 200 to 300 Å. Sepabeads SP-700 has a surface area of about 1200 m²/g, a pore volume of about 2.1 ml/g, and a pore area in the range from 90 to 100 Å. Both of Diaion HP-20 and Sepabeads SP-700 can reach up to about 100% of recovery. Sepabeads SP-700 is generally more preferred.

In step (d) of the process, the resins containing conjugated estrogens from step (c) is eluted with a solvent in an amount sufficient to separate conjugated estrogens and resins and then obtain the extract conjugated estrogens. The elution solvents suitable for the present invention can be any water-miscible solvents, preferably, lower alkanols, such as methanol and ethanol, and lower aliphatic ketones, such as acetone. The term “lower” means that the compounds contain up to 8 carbon atoms.

In order to facilitate a greater understanding of the invention, the following examples are set forth for illustration purposes only and are not to be construed as limits on the present invention.

### WORKING EXAMPLES

### Example 1

45 Liters of original pregnant mare urine (PMU) was collected with a pH value in the range from 7 to 8 and then the pH was adjusted to 11 with ION NaOH solution and filtrated. 10.5 Liters of NaOH-treated PMU was taken and contacted 20 ml of Sepabeads SP-700. 230 ML of the treated solution was taken and eluted with 230 ml of ethanol (flow rate: 200-600 ml/hr). 137.004 Mg of extracted conjugated estrogens were obtained with about 100% recovery.

### Example 2

The procedures of Example 1 were repeated except NaOH was replaced with KOH and the pH after treatment was in the range of 9-11. Recovery: 93.53%.

### Example 3

The procedures of Example 1 were repeated except NaOH was replaced with Na₂CO₃ and the pH after treatment was in the range of 9-11. Recovery: 85.44%.

### Example 4

The procedures of Example 1 were repeated except NaOH was replaced with K₂CO₃ and the pH after treatment was in the range of 9-11. Recovery: 87.14%.

The working examples illustrate that the process of the present invention can reach a much higher recovery than that of the prior art.

It is understood that the examples described herein are for illustrative purposes only and that various modifications or changes in light thereof will be suggested to one skilled in the art and are to be included in the spirit of this application and scope of the appended claims.

1. A process for the preparation of conjugated estrogens from pregnant mare urine (PMU), comprising the steps of
(a) treating PMU material with an alkaline solvent;
(b) filtrating the treated PMU material to remove mucilaginous substances and solids;
(c) contacting the filtered PMU resultant with a sufficient quantity of polystyrene absorber resins; and
(d) eluting the resins with a water-miscible solvent to separate conjugate estrogens and resins and then obtaining crude extract conjugated estrogens.

2. The process of claim 1, wherein the alkaline solvents are selected from alkali metal or alkaline earth metal hydroxides and carbonates.

3. The process of claim 2, wherein the alkaline solvents are selected from sodium hydroxide, potassium hydroxide, sodium carbonate and potassium carbonate.

4. The process of claim 3, wherein the alkaline solvent is NaOH.

5. The process of any of claims 1 to 4, wherein the pH value of the PMU solution after treatment with the alkaline solvent is in the range of 9-11.

6. The process of claim 1, wherein the filtration is carried out by centrifugation.

7. The process of claim 1, wherein the polystyrene absorber resins contain the moiety of following structural formula:

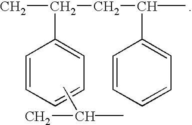

8. The process of claim 1, wherein the polystyrene absorber resin has surface area about 600 m²/g, pore volume about 1.5 ml/g and pore area in the range from 20 to 300 Å.

9. The process of claim 1, wherein the polystyrene absorber resin has surface area about 1200 m²/g, pore volume about 2.1 ml/g and pore area in the range from 90 to 100 Å.

10. The process of claim 1, wherein the elution solvents are selected from lower alkanols and lower aliphatic ketones.

11. The process of claim 10, wherein the elution solvents are selected from methanol, ethanol and acetone.
